(19) United States
(12) Patent Application Publication
(10) Pub. No.: US 2021/0268004 A1
(43) Pub. Date: Sep. 2, 2021

(54) COMPOSITION CONTAINING BECLOMETHASONE FOR THE PREVENTION AND THE TREATMENT OF BACTERIAL PROSTATITIS AND VAGINITIS

(71) Applicant: SOFAR S.P.A., Trezzano Rosa (IT)

(72) Inventor: Andrea BIFFI, Trezzano Rosa (IT)

(21) Appl. No.: 17/262,154

(22) PCT Filed: Jul. 26, 2019
(86) PCT No.: PCT/IB2019/056407

§ 371 Date: Jan. 21, 2021

(30) Foreign Application Priority Data
Jul. 26, 2018 (IT) 102018000007527

(51) Int. Cl. A61K 31/573; A61K 9/00; A61K 31/167; A61K 38/39; A61K 45/06; A61K 31/65; A61K 31/737; A61K 31/728; A61K 31/727; A61K 31/496; A61K 31/7052; A61K 31/505; A61K 31/4164; A61K 36/45; A61K 36/28; A61K 36/16; A61P 13/08

(52) U.S. Cl. CPC A61K 31/573; A61P 13/08; A61K 31/167; A61K 38/39; A61K 45/06; A61K 31/65; A61K 31/737; A61K 31/728; A61K 31/727; A61K 31/496; A61K 31/7052; A61K 31/505; A61K 31/4164; A61K 36/45; A61K 36/28; A61K 36/16; A61K 9/0014

(57) ABSTRACT

The present invention regards a composition C1 in a form for topical administration comprising beclomethasone or a derivative thereof for use in a method for treating a disease, symptom and/or disorder deriving from an inflammation of the urogenital system and the lower urinary tract, in particular prostatitis of bacterial origin, inflammatory and painful symptoms associated with said bacterial prostatitis, vaginitis and inflammatory and painful symptoms associated with said vaginitis. Furthermore, the present invention regards a combination C for use in the aforementioned treatment method comprising said composition C1, comprising beclomethasone or a derivative thereof, and a composition C2 comprising a different active ingredient.

The present invention regards a composition C1 in a form for topical administration comprising beclomethasone or a derivative thereof, preferably beclomethasone dipropionate, for use in a method for treating a disease, symptom and/or disorder deriving from an inflammation of the urogenital system and the lower urinary tract, in particular prostatitis of bacterial origin, painful symptoms associated with said bacterial prostatitis, vaginitis and painful symptoms associated with said vaginitis. Furthermore, the present invention regards a combination C for use in the aforementioned treatment method comprising said composition C1, comprising beclomethasone or a derivative thereof, and a composition C2 comprising a different active ingredient.

Prostatitis and vaginitis are two inflammations regarding the prostate and the vagina, two organs close to the rectum.

At this level, if not promptly treated, an inflammatory process causes an algesia also referring to the rectal canal or the pelvic floor area, which may also be associated to inflammation of the rectal canal, with an ensuing painful symptom.

The prostate is a gland that is part of the male reproduction system, similar to a chestnut in terms of shape and size, weighing about 20 g.

It is located immediately beneath the bladder and before the rectum and it surrounds the first part of the urethra.

The prostatitis consists in an inflammatory condition of the prostate, a bacterial or simultaneous with a bacterial infection.

According to the National Institutes of Health (NIH) there are four types of prostatitis: acute bacterial prostatitis (non-chronic) (type I), chronic bacterial prostatitis (type II), chronic a bacterial prostatitis or chronic pelvic pain syndrome, abbreviated as CP/CPPS (chronic prostatitis/chronic pelvic pain syndrome) (type III) and asymptomatic prostatitis (type IV).

Acute bacterial prostatitis is the quick and sudden inflammation of the prostate, caused by bacteria. The bacteria that cause acute bacterial prostatitis can be: the bacteria that causes urinary infections, the bacteria that cause sexually transmitted infections (e.g. chlamydia or gonorrhoea), the bacteria that generally live in the rectum (e.g. Escherichia coli). The acute prostatitis of bacterial origin can thus also originate from: an epididymitis (inflammation of the epididymis), phimosis (abnormal tightness of the foreskin), an obstruction of the bladder neck, a perpendicular injury of the perineum (area between the scrotum and the anus) or a prostate biopsy.

Acute bacterial prostatitis can be easily diagnosed by analysing white cells and bacteria in urine.

Acute bacterial prostatitis is manifested by typical signs of genitourinary infection, such as shivers, fever, pain at the lower part of the back and in the genital area, urinating problems, pain during sexual intercourse and pain when defecating.

Acute bacterial prostatitis is an emergency condition can lead to various complications if untreated, or treated late, such as: chronicity of the inflammation at prostate level, transmission of bacterial infections to close anatomical structures such as testicles, development of conditions such as epididymitis and orchitis, transfer of bacterial infections to the blood and with ensuing development of bacteraemia or sepsis, formation of a prostate abscess and inability to urinate.

Currently, the acute bacterial prostatitis therapy, established based on the response of the cultures and the correlated antibiograms, provides for the administration of targeted antibiotics.

Chronic bacterial prostatitis is relatively rare and it occurs when the bacteria find a place where they can survive in the prostate. Men suffer recurrent infections to the urinary tract, which seem to disappear but then return due to the very bacteria. Currently, the treatment requires the use of antibiotics for an extended period of time. However, antibiotics are not always able to eliminate this type of prostatitis given the difficulty for the antibiotic to perfuse the prostate at bactericidal amounts.

The treatment of both acute and chronic bacterial prostatitis may also provide for the use of anti-inflammatories, in particular NSAIDs (Non-Steroidal Anti-Inflammatory Drugs) or steroidal anti-inflammatories, combined with antibiotics with the aim of treating the inflammation and improving correlated painful symptoms.

Even herbal products could be used with the aim of reducing the inflammation, but they are generally poorly effective.

As known, the use of antibiotics causes unwanted adverse effects to the patients, such as stomach-ache, nausea, vomit and diarrhoea, especially if used over extended periods of time. Furthermore, the excessive use of antibiotics can lead to the development of a resistance by the pathogen bacteria against the antibiotic (antibiotic-resistance).

Even the use of steroidal anti-inflammatories (corticosteroids), if administered systemically (e.g. orally), causes unwanted adverse effects to the patients, due to the interference thereof with the homeostasis of the organism, such as: hypertension, water retention, hyperglycaemia, loss of potassium, osteoporosis, muscle hypertrophy, capillary fragility, delayed healing of wounds, hyperlipidaemia, accumulation of adipose tissue at facial, neck and abdomen level, gastroduodenal ulcers, increase of blood coagulability, blood alterations, euphoria and insomnia. Furthermore, in case of extended treatment corticosteroids tend to inhibit the production of similar natural hormones by the suprarenal glands, thus causing suprarenal insufficiency condition, which occurs with consequences, even serious, especially upon suspending the therapy. Another important adverse effect of extended use of corticosteroids lies in their immunosuppressive action, which increases the susceptibility to infections.

Vaginitis is an acute or chronic inflammation of the vagina which can cause secretion, itchiness and pain which are a source of considerable discomfort for women.

It is usually caused by a change to the normal bacterial balance or an infection. The three main causes of vaginitis are bacterial infections (bacterial vaginitis), yeast infections or fungal infections (vaginal candidiasis or vaginitis of fungal origin) or a protozoan that causes trichomoniasis (Trichomonas vaginalis) (non-bacterial vaginitis). A woman may suffer from several infections simultaneously. Furthermore, low levels of oestrogens, such as for example after menopause, may facilitate vaginitis, thus causing an alteration of the vaginal microbiota and vaginal atrophy, which facilitate and infective and non-infective vaginitis.

Untreated vaginal infections may lead to further complications, especially as concerns pregnant women, including premature birth, postpartum infections, clinically apparent and subclinical pelvic inflammatory disease, post-surgical complications (post-abortion, hysterectomy, caesarean section complications), increase of vulnerability to HIV infection and, possibly, infertility.

The treatment of vaginitis is subordinated to the cause that originates it. Antibiotics, to be taken systematically, such as metronidazole and tinidazole, or to be applied directly in situ for an immediate pharmacological activity (e.g. clindamycin) are currently the first-choice drugs when it comes to treating bacterial vaginitis.

In case of proven Candida (vaginitis of fungal origin) infection, this is currently treated using specific antifungal drugs, such as itraconazole, clotrimazole, cancidas and anidulafungin.

In case of atrophic vaginitis (non-bacterial vaginitis), depending on an alteration of the hormonal structure, oestradiol—possibly combined with norethindrone, esterified oestrogens or Estropipate—is prescribed so as to restore the hormone levels and hence reduce the painful symptoms of vaginal inflammation.

Furthermore, anti-inflammatory drugs, in particular steroidal anti-inflammatories (corticosteroids) are prescribed for treating vaginitis inflammatory symptoms and reduce the related pain.

Furthermore, herbal products could be used with the aim of reducing the inflammation, but they are generally poorly effective.

As mentioned above, both the use of antibiotics and the use of steroidal anti-inflammatories to treat prostatitis cause unwanted adverse effects to the patients.

Thus, there is high demand from the society for appropriate methods for treating a disease, symptom and/or disorder deriving from inflammations of the urogenital system and the lower urinary tract, in particular prostatitis of bacterial origin, painful symptoms or inflammatory conditions associated with said bacterial prostatitis, and vaginitis and painful symptoms or inflammatory conditions associated with said vaginitis.

As described above, the compositions (pharmaceutical compositions, medical device compositions, plant product compositions) currently available on the market for use in methods for treating prostatitis of bacterial origin, painful and/or inflammatory symptoms associated with said bacterial prostatitis, vaginitis and painful symptoms or inflammatory conditions associated with vaginitis, are at times ineffective in many subjects or resolute only on the short term with occurrence of relapses over time. Furthermore, some compositions can cause adverse effects, in particular those comprising antibiotics and steroidal anti-inflammatories taken systemically, e.g. orally.

The technical problem addressed and resolved by the present invention lies in providing a valid solution for the effective, quick and low or zero adverse effects treatment of diseases, symptoms and/or disorders deriving from inflammations of the urogenital system and of the lower urinary tract, in particular, prostatitis of bacterial origin, inflammatory conditions and painful symptoms associated with said bacterial prostatitis, and vaginitis and painful symptoms or inflammatory conditions associated with said vaginitis.

In order to overcome said technical problems, the present invention provides a composition comprising, as active ingredient, beclomethasone or a derivative thereof, in particular beclomethasone dipropionate, capable of effectively and quickly treating the diseases, symptoms and/or disorders deriving from inflammations of the urogenital system and of the lower urinary tract, in particular prostatitis of bacterial origin (acute or chronic), preferably acute prostatitis of bacterial origin, painful symptoms associated with said bacterial prostatitis, and vaginitis and painful symptoms associated with said vaginitis.

Furthermore, the present invention provides a composition for use in said treatment method with scarce adverse effects present in the treatments of the prior art, that are easy to prepare and economically advantageous.

Lastly, the present invention provides a composition for use in said treatment method that can be administered combined with other active ingredients present in the prior art with the aim of increasing the effectiveness of the treatment.

These and other objects, which will be clear from the detailed description that follows, are attained by the compositions and by the combinations of the present invention due to the technical characteristics claimed in the attached claims.

Following an intense research and development phase, the Applicant found out that the topical administration (vaginal or rectal) of the composition according to the present invention, comprising beclomethasone or a derivative thereof, in particular beclomethasone dipropionate, capable of effectively and quickly treating the diseases, symptoms and/or disorders deriving from inflammations of the urogenital system and of the lower urinary tract, in particular prostatitis of bacterial origin (acute or chronic), preferably acute prostatitis of bacterial origin, painful symptoms associated with said bacterial prostatitis, vaginitis and painful symptoms associated with said vaginitis.

Said pharmacological activity of beclomethasone, or a derivative thereof, for therapeutic treatment could—on first hypothesis—be due to the resolution of the local urogenital inflammatory factor and—on second hypothesis—due to the resolution of the hyperactivity condition of secondary perineum muscles in phlogistic condition.

Beclomethasone (CAS 4419-39-0, IUPAC name (8S,9R,10S,11S,13S,14S,16S,17R)-9-chloro-11,17-dihydroxy-17-(2-hydroxyacetyl)-10,13,16-trimethyl-6,7,8,11,12,14,15,16-octahydrocyclopenta[a]phenanthren-3-one), such as steroids in general, acts in the treatment of inflammatory diseases due to its capacity to regulate the inflammatory process at molecular level by means of gene regulation, in particular by inhibiting the synthesis of prostaglandins and leukotrienes, potent inflammation mediators.

In particular, beclomethasone dipropionate (abbreviated as BDP, CAS 4419-39-0, IUPAC name (8S,9R,10S,11S,13S,14S,17R)-9-Chloro-11-hydroxy-10,13,16-trimethyl-3-oxo-17[2-(propanoyloxy]-6,7,8,11,12,13,14,15,16,17-dodecahedron-3H-cyclopenta[a]phenanthren-17-yl]-2-oxoethyl] propionate), belonging to the class of second generation topical steroids (ATC code: A07EA—locally acting corticosteroids), is a synthetic glucocorticoid with a potent anti-inflammatory and immunosuppressive activity characterised by low adverse effects. In particular, BDP is subjected to a quick and extended conversion into beclomethasone-17-monopropionate (17-BMP) during the absorption. In vitro, 17-BMP shows an affinity for the human glucocorticoid receptor which is about 25 times that of beclomethasone dipropionate.

Beclomethasone dipropionate is currently used in the treatment of bronchial asthma, in the post-surgical treatment of nasal polyps, in seasonal allergic rhinitis and in chronic vasomotor rhinitis. Furthermore, BDP is used by means of topical administration in skin diseases of allergic origin and by means of rectal administration in the treatment of inflammatory diseases of the intestine which involves the terminal ileum and the ascending colon (Crohn's disease, ulcerative colitis). Rectal treatment with BDP was almost without the adverse effects typical of steroids.

Administered topically, BDP conducts its anti-inflammatory activity locally, without exercising a significant systemic activity; this due to the quick metabolic inactivation of the drug and its active metabolites after absorption. As a matter of fact, in vitro and in vivo kinetic and metabolism studies—both in humans and animals—on BDP administered in various ways revealed scarce or no systemic activity of beclomethasone dipropionate after administering appropriate formulations for oral or topically or rectally not only due to the limited absorption but also the quick inactivation of the drug during the first hepatic pass metabolism. Furthermore, BDP or the metabolites thereof are not accumulated in the tissues.

There are limited scientific researches on beclomethasone administered topically in the treatment of inflammatory symptom of the lower urinary tract. In an observatory study cited by Bozzini G et al. (BMC Urology 2016, 16: 25-32) the effectiveness and safety of beclomethasone dipropionate on the inflammation symptoms of the lower urinary tract, such as pelvic pain, urinating problems and sexual dysfunction were examined in a population of 180 patients treated with beclomethasone dipropionate in rectal suppositories, most of whom suffered from non-bacterial prostatitis (85%). An antibiotic cycle was prescribed to the patients subject of the study with bacterial infection (positive seminal fluid and/or positive urine cultures) prior to cure with BDP or during cure with BDP.

Furthermore, Fuccio L. et al. (Alimentary Pharmacology and Therapeutics, 2011; 34: 628-637) cite a double-blind vs placebo randomised clinical trial which analysis the effect of BDP administered topically (topically through the rectum) in the prevention and treatment of proctopathy induced by radiations in patients subjected to radiotherapy for prostate cancer.

Thus, currently there has not been hypothesised the use of beclomethasone or a derivative thereof, in particular betamethasone dipropionate, by means of topical administration (vaginal or rectal) in a method for treating prostatitis of bacterial origin (acute or chronic), preferably acute prostatitis of bacterial origin (non-chronic), painful symptoms associated with said bacterial prostatitis, vaginitis and painful symptoms associated with said vaginitis.

Furthermore, beclomethasone dipropionate administered topically (preferably through the rectum or vagina) is almost without adverse effects, thanks to its specific pharmacokinetics described above (limited systemic absorption and high first hepatic pass metabolism).

Forming an object of the present invention is a composition C1 (in short composition C1 of the invention) in a form for topical administration (vaginal topic or rectal topic depending on the disease) comprising

(i) a mixture M comprising, a or alternatively, consisting of beclomethasone or a derivative thereof, and, optionally,

(ii) at least one pharmaceutical grade additive and/or excipient.

wherein said composition (or pharmaceutical composition or a medical device composition) is for use in a method for treating a disease, symptom and/or disorder deriving from an inflammations of the urogenital system and of the lower urinary tract, wherein said inflammation of the lower urinary tract is selected from among prostatitis of bacterial origin (acute and/or chronic), an inflammatory condition and a painful symptom associated with said prostatitis of bacterial origin, a vaginitis (of bacterial or fungal or non-bacterial origin) and a painful symptom associated with said vaginitis.

In a preferred embodiment, said prostatitis of bacterial origin is an acute prostatitis of bacterial origin (non-chronic) and, thus, said painful and inflammatory symptom associated with said prostatitis of bacterial origin is a painful and inflammatory symptom associated with an acute prostatitis of bacterial origin (non-chronic).

In an alternative embodiment, said prostatitis of bacterial origin is a chronic prostatitis of bacterial origin and, thus, said painful and inflammatory symptom associated with said prostatitis of bacterial origin is a painful and inflammatory symptom associated with a chronic prostatitis of bacterial origin.

Preferably, said painful and inflammatory symptom associated with said prostatitis of bacterial origin (acute and/or chronic), is selected from among urinary problems, pain during sexual intercourse, painful ejaculation, pain when defecating, sense of unwellness during intestinal movements and other known symptoms.

In the context of the present invention, the expression urinating problems is used to indicate both the typical symptoms of overactive bladder (OAB) (storage/irritative symptoms) such as urgency, i.e. the urgent sensation of need to urinate, frequency, i.e. a short period of time between the urinating need intervals, nocturia, i.e. the need to urinate two or more times during the night, urgency/incontinence, i.e. a sudden, intense need to urinate followed by uncontrolled leakage of urine, voiding/obstructive symptoms such as hesitation, i.e. an unusually longer waiting at the beginning of the exit of urine, weak and never straight flow of urine, need to strain when urinating, chronic urine retention, i.e. the urine is not fully transferred from the from the bladder thus causing a more often need to urinate, overflowing or paradoxical incontinence, i.e. the urine uncontrollably overflows from a full bladder even though normal urination can be difficult to start.

Preferably, said painful and inflammatory symptom associated with vaginitis (of bacterial or fungal or non-bacterial origin) is selected from among burning sensation, reddening, itchiness, swelling of the external genitalia, pain and contractions of the pelvic floor, pain during sexual intercourse (dyspareunia), pain when urinating, vaginal irritation, change of colour, odour and/or amount of vaginal secretions, slight bleeding and other known symptoms.

In the context of the present invention, the expression derivatives of beclomethasone is used to indicate its prodrugs, solvates, metabolites, precursors and salts, or, alternatively, its analogues such as for example betamethasone, betamethasone benzoate, betamethasone dipropionate, betamethasone propionate, betamethasone valerate.

In a preferred embodiment, in the context of the present invention, the expression “a derivative thereof” referring to a beclomethasone is used to indicate a prodrug of beclomethasone and the respective salts thereof. A beclomethasone prodrug is, for example, a mono- or di-ester of an alkyl or aryl acid of beclomethasone, wherein said alkyl acid is a linear, branched, cyclic or acyclic alkyl acid, having a number of carbons comprised between C2 and C12, preferably between C3 and C7, and said aryl acid is an aryl acid having a number of carbons between C7 and C20, preferably between C7 and C14; preferably said beclomethasone derivative or prodrug is beclomethasone dipropionate (di-ester of propyl acid of beclomethasone, as the beclomethasone prodrug).

The prodrug is a biologically inactive molecule which, once introduced into the organism, undergoes chemical transformation, generally by enzymes, which activate it. Thus, the prodrug is a precursor of the active ingredient. In the present invention, for example, the active ingredient is beclomethasone and the prodrug is beclomethasone dipropionate.

In a preferred embodiment, in the composition according to the invention, said (i) mixture M comprises or, alternatively, consists of beclomethasone dipropionate or its solvates, products of hydrolysis, metabolites, precursors and the salts thereof; preferably beclomethasone dipropionate. Advantageously, the esterification of beclomethasone with propionic acid, or a different alkyl or aryl acid, increases the lipophilia of beclomethasone and facilitates the absorption thereof by the mucosa (rectal or vaginal) and the phospholipid membrane. After administering the esterified beclomethasone (prodrug) is biotransformed by the organism in the active form thereof, such as beclomethasone.

In an alternative embodiment. in the context of the present invention, the expression “a derivative thereof” referring to a beclomethasone is used to indicate an analogue compound of beclomethasone or a prodrug, solvate, products of hydrolysis, metabolites, precursor, salt of said analogue. An example of analogue of beclomethasone is betamethasone. Betamethasone (IUPAC name (8S,9R,10S,11S,13S,14S,16S,17R)-9-fluoro-11,17-dihydroxy-17-(2-hydroxyacetyl)-10,13,16-trimethyl-6,7,8,11,12,14,15,16-octahydrocyclopenta[a]phenanthren-3-one and CAS nr 378-44-9) is a compound belonging to the class of glucocorticoids having a structure similar to beclomethasone with the sole difference lying in the presence of a fluoro atom instead of the chloro atom of beclomethasone. Examples of betamethasone prodrugs are: betamethasone benzoate, betamethasone dipropionate, betamethasone propionate and betamethasone valerate.

According to a preferred aspect of the invention, the composition C1 comprises an amount by weight of beclomethasone or of a derivative thereof, preferably beclomethasone dipropionate, comprised in the range between 0.1 mg and 20 mg, preferably between 1 mg and 10 mg, more preferably 3 mg or 6 mg or 9 mg, even more preferably 3 mg or 6 mg, wherein said amounts by weight present in a single dose unit, for example a suppository, enema, pessary, ovule, foam, gel or cream.

Forming an object of the invention is a method for treating a disease, symptom and/or disorder deriving from an inflammations of the urogenital system and of the lower urinary tract, wherein said inflammation of the lower urinary tract is selected from among a prostatitis of bacterial origin (acute or chronic), preferably acute prostatitis (non-chronic) of bacterial origin, an inflammatory and painful symptom associated with said prostatitis of bacterial origin, a vaginitis (of bacterial or fungal or non-bacterial origin) and an inflammatory and painful symptom associated with said vaginitis, as defined above, by administering an effective amount of the composition C1 (or pharmaceutical composition or a medical device composition) of the present invention to a needy subject.

Preferably, the composition C1 of the invention, comprises the amounts of beclomethasone or of a derivative thereof, preferably beclomethasone dipropionate, indicated above, can be administered to said subject one or two or three times a day. For example, the composition C1 of the invention comprising 3 mg or 6 mg of beclomethasone dipropionate may be administered to a needy subject one or two or three times a day, depending on the disease, symptom and/or disorder and the severity thereof. The duration of the treatment may be comprised in a time interval ranging between 3 days to 8 weeks, preferably between 1 and 6 weeks, depending on the severity of the disease and in the fact that it is one of the aforementioned diseases (i.e. bacterial prostatitis, vaginitis or symptoms associated therewith) in acute or chronic form.

The composition C1 of the present invention can be, by way of non-limiting example, in a liquid form, such as a solution, two-phase liquid system or suspension, semi-solid form, such as gel, cream or foam, or solid form.

The composition C1 (or pharmaceutical composition or a medical device composition) of the invention can be formulated in a form appropriate for topical administration thereof, in particular topically through the rectum or topically through the vagina, such as, for example in the form of a suppository, enema, pessary, ovule, foam, gel or cream.

The composition C1 of the present invention may comprise, additionally to beclomethasone or a derivative thereof, preferably beclomethasone dipropionate, pharmaceutical grade additives and/or excipients i.e. a substance without therapeutic activity suitable for pharmaceutical use. In the context of the present invention the acceptable ingredients for pharmaceutical use comprise all auxiliary substances known to the man skilled in the art such as, by way of non-limiting example, diluents, solvents (including water, glycerine, ethyl alcohol), solubilisers, thickeners, sweeteners, flavourings, dyes, lubricants, surfactants, antimicrobials, antioxidants, preservatives, pH stabilisation buffers and the mixtures thereof.

Preferably, when the composition C1 is formulated in the form of a suspension, the additives and/or excipients can be methyl p-hydroxybenzoate, ethyl p-hydroxybenzoate, disodium salt of ethylenediaminetetraacetic acid, monobasic sodium phosphate dihydrate, dibasic sodium phosphate dodecahydrate, sodium carboxymethyl cellulose and/or purified water.

Preferably, when the composition C1 is formulated in the form of a foam, the additives and/or excipients can be methyl p-hydroxybenzoate, sodium propyl p-hydroxybenzoate, disodium salt of ethylenediaminetetraacetic acid, cetostearyl polyoxyethylene esters of sorbitan, polysorbate 20, propylene glycol, glycerides of polyoxyethylated fatty acids, isobutane, propane, butane and/or purified water.

Preferably, when the composition C1 is formulated in form of a suppository, the additives and/or excipients can be solid semi-synthetic glycerides.

Lastly, forming an object of the present invention, a pharmaceutical composition or a medical device composition comprising or, alternatively, consisting of the composition C1 of the present invention.

The expression “medical device” in the context of the present invention is used according to the meaning laid down by the Italian Legislative Decree n° 46, dated 24 Feb. 1997, i.e. it indicates a substance or another product, used alone or in combination, designated by the manufacturer to be used in humans for diagnosis, prevention, control, therapy or disease attenuation purposes, the product not exercising the main action, in or on the human body, for which it is designated, neither with pharmacological or immunology means nor by means of a metabolic process but the function thereof can be assisted by such means.

Furthermore, forming an object of the present invention is a combination C (in short combination C of the invention) for use in a method for treating a disease, symptom and/or disorder deriving from an inflammation of the urogenital system and of the lower urinary tract, wherein said inflammation of the lower urinary tract is selected from among:
a prostatitis of bacterial origin (acute or chronic) as defined above,
an inflammatory and painful symptom associated with said prostatitis of bacterial origin as defined above;
a vaginitis and
an inflammatory and painful symptom associated with said vaginitis as defined above;
wherein said combination C comprises
the composition C1 according to any one of the embodiments described above, and
a composition C2 comprising or, alternatively, consisting of: a glycosaminoglycan such as hyaluronic acid, chondroitin sulphate, dermatan sulphate, keratan sulphate, heparan sulphate and heparin or collagen, an antibiotic, an alpha blocker/lytic, a urinary antispasmodic, a 5-alpha reductase inhibitor, an NSAID (Nonsteroidal anti-inflammatory drug), paracetamol (alternatively called acetaminophen, N-acetyl-para-aminophenol), cortisone, an oestrogen, an antihistamine, an analgesic, an antimycotic, a plant product (botanicals) and the mixtures thereof.

In an embodiment of the combination C of the invention, said composition C2 does not comprise or, alternatively, does not consist of an antibiotic when said combination C is for use in a method for treating the painful symptom associated with said prostatitis of bacterial origin and the composition C1 comprises the (i) mixture M comprising, a or alternatively, consisting of beclomethasone dipropionate and said composition C1 is formulated in form of a suppository.

In an embodiment of the combination C of the invention, said prostatitis of bacterial origin is an acute prostatitis of bacterial origin (non-chronic) and, thus, said inflammatory and painful symptom associated with said prostatitis of bacterial origin is an inflammatory and painful symptom associated with an acute prostatitis of bacterial origin (non-chronic).

In an embodiment of the combination C of the invention, said prostatitis of bacterial origin is a chronic prostatitis of bacterial origin and, thus, said inflammatory and painful symptom associated with said prostatitis of bacterial origin is an inflammatory and painful symptom associated with a chronic prostatitis of bacterial origin.

Preferably, when said composition C2 comprises or, alternatively, consists of an antibiotic, said antibiotic is selected from among a quinolone, preferably ciprofloxacin (IUPAC name 1-Cyclopropyl-6-Fluoro-1,4-dihydro-4-oxo-7-(1-piperazinyl)-3-quinolinecarboxylic acid, CAS 85721-33-1), levofloxacin (IUPAC name (S)-9-fluoro-2-3-dihydro-methyl-10-(4-methylpiperazin-yl)-7-oxo-7H-pyrido(1,2,3-de)-1,4-benzoxazine-6-carboxylic acid, CAS 100986-85-4), ofloxacin (CAS 82419-36-1), a cephalosporin preferably cefalexin (IUPAC name (6R,7R)-7-[[(2R)-2-amino-2-phenylacetyl]amino]-3-methyl-8-oxo-5-thia-1-azabicyclo[4.2.0]oct-2-ene-2-carboxylic acid, CAS 15686-71-2), a macrolide, preferably azithromycin (IUPAC name (2R,3S,4R,5R,8R,10R,11R,12S,13S,14R)-11-[(2S,3R,4S,6R)-4-(dimethylamino)-3-hydroxy-6-methyloxan-2-yl]oxy-2-ethyl-3,4,10-trihydroxy-13-[(2R,4R,5S,6S)-5-hydroxy-4-methoxy-4,6-dimethyloxan-2-yl]oxy-3,5,6,8,10,12,14-heptamethyl-1-oxa-6-azacyclopentadecan-15-one, CAS 83905-01-5), a tetracycline, preferably doxycycline (IUPAC name (4S,4aR,5S,5aR,6R,12aS)-2-[amino(hydroxy)methylene]-4-(dimethylamino)-5,10,11,12a-tetrahydroxy-6-methyl-4a,5,5a,6-tetrahydro-4H-tetracene-1,3,12-trione, CAS 564-25-0) or tetracycline (IUPAC name 4S,6S,12aS)-4-(dimethylamino)-1,4,4a,5,5a,6,11,12a-octahydro-3,6,10,12,12a-pentahydroxy-6-methyl-1,11-dioxonaphthacene-2-carboxamide, CAS 60-54-8), a sulfamide, preferably trimethoprim (IUPAC name 5-[(3,4,5-trimethoxyphenyl)methyl]pyrimidine-2,4-diamine, CAS 738-70-5), and a nitroimidazole, preferably metronidazole (IUPAC name 2-(2-methyl-5-nitro-1H-imidazole-1-yl)ethanol, CAS 443-48-1).

Preferably, when said composition C2 comprises or, alternatively, consists of an alpha-blocker or alpha-lytic, said alpha-blocker or alpha-lytic is selected from among doxazosin (IUPAC name [4-(4-amino-6,7-dimethoxyquinazolin-2-yl)piperazin-1-yl](2,3-dihydro-1,4-benzodioxin-3-yl)methanone, CAS 74191-85-8), terazosin (IUPAC name [4-(4-Amino-6,7-dimethoxyquinazolin-2-yl)piperazin-1-yl]-(2,3-dihydro-1,4-benzodioxin-3-yl)methanone, CAS 63590-64-7), tamsulosin (IUPAC name 5-[(2R)-2-[2-(2-ethoxyphenoxy)ethylamino]propyl]-2-methoxybenzenesulfonamide, CAS 106133-20-4), silodosin (IUPAC name 1-(3-hydroxypropyl)-5-[(2R)({2-[2-[2-(2,2,2-trifluoroethoxy)phenoxy]ethyl}amino)propyl]indole-7-carboxamide, CAS 160970-54-7).

Preferably, when said composition C2 comprises or, alternatively, consists of a urinary antispasmodic said urinary antispasmodic is oxybutynin (IUPAC name 4-(diethylaminobut-2-ynyl 2-cyclohexyl-2-hydroxy-2-phenylacetate, CAS 5633-20-5).

Preferably, when said composition C2 is a 5-alpha reductase inhibitor, said 5-alpha reductase inhibitor is selected from among finasteride (IUPAC name N-(1,1-dimethylethyl)-3-oxo-(5α,17β)-4-azaandrost-1-ene-17-carboxamide, CAS 98319-26-7) and dutasteride (IUPAC name (1S,3aS,3bS,5aR,9aR,9bS,11aS)-N-[2,5-bis(trifluoromethyl)phenyl]-9a,11a-dimethyl-7-oxo-1,2,3,3a,3b,4,5,5a,6,9b,10,11-dodecahydroindeno[5,4-f]quinoline-1-carboxamide, CAS 164656-23-9).

Preferably, when said composition C2 comprises or, alternatively, consists of an NSAID (Nonsteroidal anti-inflammatory drug) said NSAID is ibuprofen (IUPAC name (RS)-2-[4-(2-methylpropyl)phenyl]propanoic acid, CAS 15687-27-1).

Preferably, when said composition C2 comprises or, alternatively, consists of an antimycotic, said antimycotic is selected from among fluconazole (IUPAC name; 2-(2,4-difluorophenyl)-1,3-bis(1,2,4-triazol-1-yl)propan-2-ol, CAS 86386-73-4), terconazole (IUPAC name 1-[4-[[(2R,4S)-2-(2,4-dichlorophenyl)-2-(1,2,4-triazol-1-ylmethyl)-1,3-dioxolan-4-yl]methoxy]phenyl]-4-propan-2-ylpiperazine, CAS 67915-31-5), clotrimazole (IUPAC name 1-[(2-chlorophenyl)-diphenyl-methyl]imidazole CAS 23593-75-1), miconazole (IUPAC name 1-[2-(2,4-dichlorophenyl)-2-[(2,4-dichlorophenyl)methoxy]ethyl]imidazole, CAS 22916-47-8) and butoconazole (IUPAC name 1-[4-(4-chlorophenyl)-2-(2,6-dichlorophenyl)sulfanylbutyl]imidazole, CAS 67085-13-6).

Preferably, when said composition C2 comprises or, alternatively, consists of a plant product (botanicals) or an extract or derivative thereof, said plant product is selected from among root of nettle (Urtica dioica), Ginkgo biloba, Echinacea, goldenrod (Solidago virgaurea), strawberry tree (Arbutus unedo), bearberry (Arctostaphylos uva-ursi), highbush blueberry (Vaccinium macrocarpon or cranberry), rye (Secale cereale) and Serenoa repens.

In an embodiment of the combination C of the invention, said composition C1 and said composition C2 are administered to a subject separately and in any order.

In the context of the present invention, administration to a subject separately comprises both when the composition C1 and C2 are administered to a subject in a close sequence over time (between 0 and 60 minutes) or in a non-close sequence (between 1 hour and 24 hours) and when the compositions C1 and C2 are administered to a subject at the same frequency or with different frequencies.

In this embodiment, the composition C1 is administered topically (vaginal or rectal) as defined above and the composition C2 can be administered in any form suitable for the absorption thereof by the organism, which will vary depending on the active ingredient comprised in the composition C2. In particular, the composition C2 can be administered topically (including through the vagina or rectum) or enterally, such as for example orally (or gastrointestinal) and sublingually (or buccal) parenterally.

In an alternative embodiment of the combination C of the invention, said composition C1 and said composition C2 are administered to a subject in a single formulation for topical use (vaginal or rectal). In this embodiment, the composition C can be formulated in a form suitable for topical administration (rectal or vaginal) thereof, similarly to the description regarding composition C1, such as suppository, enema, pessary, ovule, foam, gel or cream.

The expression “treatment method” in the context of the present invention is used to indicate an action, comprising the administration of a substance, or mixture of substances or combination thereof, with the aim of eliminating, reducing or preventing a pathology or disease and its symptoms or disorders.

Unless specified otherwise, the indication that a composition “comprises” one or more components or substances means that other components or substances can be present besides the one, or the ones, indicated specifically.

The composition C1 and the combination C of the present invention shall be deemed indistinctly for human or veterinarian use, i.e. as a preparation to be applied to animals by means of the uses and methods known to the man skilled in the art.

Embodiments (Frnr) of the present invention are indicated below:

FR1. A composition C1 in a form for topical administration comprising:
(i) a mixture M comprising, a or alternatively, consisting of beclomethasone or a derivative thereof, and, optionally,
(ii) at least one pharmaceutical grade additive and/or excipient.

wherein said composition is for use in a method for treating a disease, symptom and/or disorder deriving from an inflammation of the urogenital system and of the lower urinary tract, wherein said inflammation of the lower urinary tract is selected from among a prostatitis of bacterial origin, an inflammatory and painful symptom associated with said prostatitis of bacterial origin, a vaginitis and an inflammatory and painful symptom associated with said vaginitis.

FR2. The composition C1 according to FR1 for use according to any one of the preceding claims, wherein said prostatitis of bacterial origin is an acute prostatitis of bacterial origin and said inflammatory and painful symptom associated with said prostatitis of bacterial origin is an inflammatory and painful symptom associated with an acute prostatitis of bacterial origin.

FR3. The composition C1 according to FR1 or FR2, wherein said (i) mixture M comprises or, alternatively, consists of beclomethasone or betamethasone benzoate or betamethasone dipropionate or betamethasone propionate or betamethasone valerate; preferably betamethasone dipropionate.

FR4. The composition C1 according to any one of FR1 to FR3, wherein said inflammatory and painful symptom associated with said prostatitis of bacterial origin, preferably inflammatory and painful symptom associated with said acute prostatitis of bacterial origin is selected from among the group comprising, a or alternatively, consisting of: urinary problems, pain during sexual intercourse, painful ejaculation, pain when defecating and sense of unwellness during intestinal movements.

FR5. The composition C1 according to any one of FR1 to FR4, wherein said painful symptom associated with said vaginitis is selected from among the group comprising, a or alternatively, consisting of: burning sensation, reddening, itchiness, swelling of external genitalia, pain during sexual intercourse (dyspareunia), pain when urinating, vaginal irritation.

FR6. The composition C1 according to any one of FR1 to FR5, wherein the composition C1 comprises an amount by weight of beclomethasone or a derivative thereof, preferably beclomethasone dipropionate, comprised in the range between 0.1 mg and 20 mg, preferably between 1 mg and 10 mg, more preferably 3 mg or 6 mg or 9 mg.

FR7. The composition C1 according to any one of FR1 to FR6, wherein the composition C1 is formulated in form of a suppository, enema, pessary, ovule, foam, gel or cream.

FR8. A combination C for use in a method for treating a disease, symptom and/or disorder deriving from an inflammation of the urogenital system and of the lower urinary tract, wherein said inflammation of the lower urinary tract is selected from among:
a prostatitis of bacterial origin, preferably an acute prostatitis of bacterial origin;
an inflammatory and painful symptom associated with said prostatitis of bacterial origin, preferably urinary problems, pain during sexual intercourse, painful ejaculation, pain when defecating and sense of unwellness during intestinal movements;
a vaginitis and
an inflammatory and painful symptom associated with said vaginitis, preferably burning sensation, reddening, itchiness, swelling of external genitalia, pain during sexual intercourse (dyspareunia), pain when urinating, vaginal irritation;

wherein said combination C comprises
the composition C1 according to any one of claims 1 to 7, and
a composition C2 comprising or, alternatively, consisting of: a glycosaminoglycan such as hyaluronic acid, dermatan sulphate, chondroitin sulphate, keratan sulphate, heparan sulphate and heparin, collagen, an antibiotic, an alpha-blocker/lytic, a urinary antispasmodic, a 5-alpha reductase inhibitor, a NSAID (Nonsteroidal anti-inflammatory drug), paracetamol, cortisone, an estrogen, an antihistamine, an analgesic, an antimycotic, a plant product (botanicals) and the mixtures thereof; with the condition that said composition C2 does not comprise or, alternatively, does not consist of an antibiotic when said combination C is for use in a method for treating the inflammatory and painful symptom associated with said prostatitis of bacterial origin and the composition C1 comprises the (i) mixture M comprising or, alternatively, consisting of beclomethasone dipropionate and said composition C1 is formulated in form of suppository.

FR9. The combination C according to FR8 wherein said composition C1 and said composition C2 are administered separately or, alternatively, wherein said composition C1 and said composition C2 are administered in a single formulation for topical use.

FR10. The combination C according to FR9, wherein when said composition C2 comprises or, alternatively, consists of an antibiotic, said antibiotic is selected from among a quinolone, preferably ciprofloxacin, levofloxacin, ofloxacin, a cephalosporin, preferably cephalexin, a macrolide, preferably azithromycin, a tetracycline, preferably doxycycline or tetracycline, a sulfonamide, preferably trimethoprim, and a nitroimidazole, preferably metronidazole; wherein when said composition C2 comprises or, alternatively, consists of an alpha blocker or alpha-lytic, said alpha blocker or alpha-lytic is selected from among doxazosin, terazosin, tamsulosin, silodosin; wherein when said composition C2 comprises or, alternatively, consists of a urinary antispasmodic, said urinary antispasmodic is oxybutynin; wherein when said composition C2 is a 5-alpha reductase inhibitor, said 5-alpha reductase inhibitor is selected from among finasteride and dutasteride; wherein when said composition C2 comprises or, alternatively, consists of an NSAID (Nonsteroidal anti-inflammatory drug), said NSAID is ibuprofen; wherein when said composition C2 comprises or, alternatively, consists of an antimycotic, said antimycotic is selected from among fluconazole, terconazole, clotrimazole, miconazole and butoconazole; wherein when said active ingredient C2 comprises or, alternatively, consists of a plant product (botanicals), said plant product is selected from among the root of nettle, ginkgo, Echinacea, goldenrod, strawberry tree, bearberry, highbush blueberry, rye and Serenoa repens.

### EXPERIMENTAL PART

### (I) Experiment 1: Beclomethasone Dipropionate (BDP) Passage on Colorectal Mucosa

(I.I) Trial 1

(I.I.1) Purpose and Design of the Experiment

The transepithelial passage, the penetration kinetics and the distribution of beclomethasone dipropionate (BDP) included in a solid technical form (suppositories) and the acid metabolite thereof (beclomethasone acid (BMP) through the colorectal epithelium was evaluated in a preliminary trial of penetration into an epithelial tissue of the human colon reconstructed in 3D.

The trial was conducted according to the OCSE TG 428-EU approved method used for percutaneous penetration.

Table 1 below shows the doses of beclomethasone dipropionate (BDP) applied on the colorectal mucosa (area 0.5 cm²). The test was conducted at 37° C. and relative humidity >90%.

TABLE 1

| Treatment | | |
|---|---|---|
| Amount applied | Amount applied/cm² | |
| Composition A: | 20 mg suppository/cm² | Quantifying the transepithelial |
| 10 mg suppository | (=40 μg BDP) | passage of beclomethasone |
| (=20 μg BDP) | | dipropionate (BDP) and the |
| Composition B: | 40 mg suppository/cm² | metabolite acid thereof (BMP) in |
| 20 mg suppository | (=80 μg BDP) | the receptor fluid (9 h and 24 h) |
| (=40 μg BDP) | | and residual and homogenised |
| Composition C: | 40 mg suppository/cm² | tissue (at the end of exposure, |
| 10 mg + 10 mg | (=80 μg BDP) | 24 h) |
| 2 suppositories with 12-hr | | |
| intervals (= total 40 μg BDP) | | |

(I.I.2) Results

The results obtained for the quantification of beclomethasone dipropionate (BDP) and the acid form thereof (BMP) are shown in table 2 below. The analytical method allows to quantify the concentrations of BDP with an estimated LOQ of 0.2 μg/mL (LOQ: limit of quantification). In the experimental conditions adopted, no passage of BDP through the epithelium was observed. Furthermore, the BDP was not metabolised in BMP (not detected).

TABLE 2

| | Applied | | RF | | RF | | | | | | | |
|---|---|---|---|---|---|---|---|---|---|---|---|---|
| | dose | | 9 h | | 24 h | | HMG | | Residue | | | recovery |
| | BDP μg | cm² | BDP | BMP | BDP | BMP | BDP | BMP | BDP | BMP | Total | % |
| Comp A | 21 | 42 | 0 | 0 | 0 | 0 | 1 | 0 | 48 | 0 | 19 | 90 |
| Comp B | 42 | 84 | 0 | 0 | 0 | 0 | 0 | 0 | 44 | 0 | 44 | 105 |
| Comp C | 42 | 84 | 0 | 0 | 0 | 0 | 0 | 0 | 43 | 0 | 43 | 102 |

(I.II) Trial 2

(I.II.1) Purpose and Design of the Experiment

In this phase, pure beclomethasone dipropionate (BDP) is dissolved in a lipophilic liquid vehicle (glycerine-mygliol 812 (50:50) and 0.1% DMSO) at a concentration of 5 mg/mL and it was applied with the aim of understanding:
whether the technical form (suppository) is a limiting factor in the bioavailability of BDP in the adopted experimental conditions;
whether BDP is actually converted in its acid form by the colorectal epithelium and detected by it in the fluids of the receptor;

a very high amount of BDP (conditions D) was also applied with the aim of understanding whether concentration is a limiting factor in the metabolism of I BDP in acid form.

(I.II.2) Results

Table 3 below shows the doses of BDP applied on the colorectal mucosa (area 0.5 cm²). Like in the first trial, the test was conducted at 37° C. and relative humidity >90%.

TABLE 3

| Treatment | | |
|---|---|---|
| Amount applied | Amount applied/cm² | |
| BDP A: 15 μL | 150 μg | Quantifying the transepithelial passage of |
| (=75 μg BDP) | BDP/cm² | beclomethasone dipropionate (BDP) and the |
| BDP B: 15 μL + 15 μL | 300 μg | metabolite acid thereof (BMP) in the receptor |
| (=75 μg + 75 μg BDP, | BDP/cm² | fluid (9 h and 24 h) and residual and |
| with a 9-hr interval) | | homogenised tissue (at the end of |
| BDP C: 30 μL | 300 μg | exposure, 24 h) |
| (=150 μg BDP) | BDP/cm² | |
| BDP D: 6 mg BDP with | 12 μg | Quantifying the transepithelial passage of |
| addition of 30 μL vehicle | BDP/cm² | beclomethasone dipropionate (BDP) and the |
| directly on tissue | | metabolite acid thereof (BMP) in the receptor |
| | | fluid (24 h and 48 h) and residual and |
| | | homogenised tissue (at the end of exposure, |
| | | 48 h) |

Table 4 below shows the doses of BDP applied on the colorectal mucosa (area 0.5 cm²). Like in the first phase, the test was conducted at 37° C. and relative humidity >90%.

TABLE 4

| | Applied | | RF | | RF | | | | | | | |
|---|---|---|---|---|---|---|---|---|---|---|---|---|
| | dose | | 9 h | | 24 h | | HMG | | Residue | | | recovery |
| | BDP μg | cm² | BDP | BMP | BDP | BMP | BDP | BMP | BDP | BMP | Total | % |
| BDP A | 75 | 150 | 0.19 | 0.22 | 0.21 | 0.19 | 0.67 | 0.40 | 147.64 | 0.30 | 149.81 | 200 |
| BDP B | 150 | 300 | 0.23 | 0.20 | 0.25 | 0.20 | 1.32 | 0.40 | 130.53 | 0.40 | 133.53 | 89 |
| BDP C | 150 | 300 | 0.42 | 0.20 | 0.23 | 0.20 | 1.88 | 0.40 | 108.79 | 0.40 | 112.53 | 75 |

| | | | RF | | RF | | | | | | | |
|---|---|---|---|---|---|---|---|---|---|---|---|---|
| | | | 24 h | | 28 h | | | | | | | |
| | | | BDP | BMP | BDP | BMP | BDP | BMP | BDP | BMP | | |
| BDP D | 6000 | 12 mg | 0.20 | 0.30 | 0.20 | 0.20 | 689.71 | 0.40 | 647.9 | 0.40 | 1339.30 | 22 |

The results show that:
The passage of BDP is very low (equal to or lesser than the LOQ) and it is dose-dependent: when BDP is applied at a dose of 150 μg a higher passage is observed (about 1% after 24 hours);
BDP is mainly detected in the residual fraction and partly in the homogenate of the tissue if applied at very high amounts (condition D): however, the amount of 6 mg is not realistic for the field of application used in the experimental conditions adopted (12 mg/cm²);
The acid form of BMP is formed at very low amounts (equal to or lesser than the LOQ).

(I.III) Conclusions of the Experiments 1

In conclusion, the preliminary results show that, after 24 hours, the amount of BDP penetrated into the tissue (HMG, homogenised tissue) is 1.88 μg (considering condition C, which gave the best results from an analytical standpoint), corresponding to about 1% of the amount applied. Thus, this dose can be potentially metabolised in acid form.

### (II) Experiment 2: Evaluation of the Absorption of Beclomethasone Dipropionate at the Prostate Level and Prostate Model Used

In order to evaluate the absorption of beclomethasone dipropionate (BDP) an assay based on an organoid (such as a microtissue of prostate cells reproducing the general structure of the prostate) which evaluates the passage of BDP from the medium in which the microtissue is “suspended” inside the microtissue, is used at prostate level. Then, the BDP in the medium (residual fraction) and in the microtissue is quantified. The prostate model (microtissue of prostate cells) is obtained using the method described below. The cells are seeded in Gravity Plus and the formation of the microtissue (MT) is followed by microscopic inspection: upon achieving a suitable shape and once the edge is well-formed, they will be transferred in Gravity Trap and cultured using an appropriate medium according to the experimental design and number appropriate to the dishes.

For each microtissue of prostatitis cells, an x amount of BDP is applied in the medium for 24h or 72h, for example 6 μg of BDP in the growth medium (LOQ 0.2 μg). At the end of the exposure, in order to obtain amounts of critical analytes for quantification using the provided analytical method, an n number of microtissues are collected together in a single pool (for example 15), and same case applying to the respective media: thus, the quantifiable concentration of BDP will theoretically be x*n of applied dose, for example 6 μg*15=90 μg. The samples are collected in 3 pools.

At the end of the exposure, the tissue is collected and the BDP is quantified by the analytical method validated in the medium and on the homogenates. The viability in the defined time points is measured using ATP dosage.

Tissues for histological analysis are prepared simultaneously.

Subsequently to this absorption trial on the prostate model, representing a first passage quantification step, analytical techniques, for example the MALDI, which allow to detect the BDP directly on the whole tissue (not homogenate) and thus establish whether located on the surface or in the organoid, can be plausibly used.

The mass spectrometry imaging MALDI (MALDI-IMS) is the use of matrix-assisted laser desorption ionisation as the mass spectrometry imaging technique wherein the sample, often a thin tissue section, is displaced in two dimensions while the mass spectrum is recorded. The advantages, such as simultaneously measuring the distribution of a large amount of analytes without destroying the sample, make it a method useful for the tissue-based trial.

1. A method of treating a disease, symptom and/or disorder deriving from an inflammation of the urogenital system and of the lower urinary tract, wherein said inflammation of the lower urinary tract is: i vaginitis or an inflammatory and painful symptom associated with said vaginitis or ii. prostatitis of bacterial origin or an inflammatory and painful symptom associated with said prostatitis of bacterial origin, comprising topically administering a composition comprising:
(i) a mixture M comprising, a or alternatively, consisting of beclomethasone or a derivative thereof, wherein said derivative is beclomethasone prodrug, and, optionally,
(ii) at least one pharmaceutical grade additive and/or excipient.

2. The method of claim 1, wherein said inflammation of the lower urinary tract is a prostatitis of bacterial origin or an inflammatory and painful symptom associated with said prostatitis of bacterial origin.

3. The method of claim 1, wherein said beclomethasone derivative is beclomethasone dipropionate.

4. The method of claim 1, wherein said vaginitis is of bacterial origin or of non-bacterial origin and/or of fungal origin.

5. The method of claim 1, wherein said prostatitis of bacterial origin is an acute prostatitis of bacterial origin and said inflammatory and painful symptom associated with said prostatitis of bacterial origin is an inflammatory and painful symptom associated with an acute prostatitis of bacterial origin.

6. The method of claim 1, wherein said prostatitis of bacterial origin is a chronic prostatitis of bacterial origin and said inflammatory and painful symptom associated with said prostatitis of bacterial origin is an inflammatory and painful symptom associated with a chronic prostatitis of bacterial origin.

7. The method of claim 1, wherein said inflammatory and painful symptom associated with said acute or chronic prostatitis of bacterial origin is selected from among the group comprising, a or alternatively, consisting of: urinary problems, pain during sexual intercourse, painful ejaculation, pain when defecating and sense of unwellness during intestinal movements.

8. The method of claim 1, wherein said painful symptom associated with said vaginitis is selected from among the group comprising, a or alternatively, consisting of: burning sensation, reddening, itchiness, swelling of external genitalia, pain during sexual intercourse (dyspareunia), pain when urinating, vaginal irritation.

9. The method of claim 1, wherein the composition C1 comprises, for single dose unit, an amount by weight of beclomethasone or a derivative thereof, wherein said derivative is a prodrug, preferably beclomethasone dipropionate, comprised in the range between 0.1 mg and 20 mg, preferably between 1 mg and 10 mg, more preferably 3 mg or 6 mg or 9 mg.

10. The method of claim 1, wherein the composition is formulated in form of a suppository, enema, pessary, ovule, foam, gel or cream.

11. A method of treating a disease, symptom and/or disorder deriving from an inflammation of the urogenital system and of the lower urinary tract, wherein said inflammation of the lower urinary tract is selected from among:
a prostatitis of bacterial origin;
an inflammatory and painful symptom associated with said prostatitis of bacterial origin, preferably urinary problems, pain during sexual intercourse, painful ejaculation, pain when defecating and sense of unwellness during intestinal movements;
a vaginitis and
an inflammatory and painful symptom associated with said vaginitis, preferably burning sensation, reddening, itchiness, swelling of external genitalia, pain during sexual intercourse (dyspareunia), pain when urinating, vaginal irritation;
comprising administering a combination to a patient, wherein said combination comprises
a composition C1, comprising beclomethasone or a derivative thereof, wherein said derivative is beclomethasone prodrug, and, optionally, at least one pharmaceutical grade additive and/or excipient, and
a composition C2 comprising or, alternatively, consisting of: a glycosaminoglycan, collagen, an antibiotic, an alpha blocker/lytic, a urinary antispasmodic, a 5-alpha reductase inhibitor, an NSAID (Nonsteroidal anti-inflammatory drug), paracetamol, cortisone, an oestrogen, an antihistamine, an analgesic, an antimycotic, a plant product (botanicals) and the mixtures thereof.

12. The method of claim 11, wherein said composition C1 and said composition C2 are administered separately or, alternatively, wherein said composition C1 and said composition C2 are administered in a single formulation for topical use.

13. The method of claim 12, wherein when said composition C2 comprises or, alternatively, consists of a glycosaminoglycan, said glycosaminoglycan is selected from among hyaluronic acid, dermatan sulphate, chondroitin sulphate, keratan sulphate, heparan sulphate and heparin; wherein when said composition C2 comprises or, alternatively, consists of an antibiotic, said antibiotic is selected from among a quinolone, preferably ciprofloxacin, levofloxacin, ofloxacin, a cephalosporin, preferably cephalexin, a macrolide, preferably azithromycin, a tetracycline, preferably doxycycline or tetracycline, a sulfonamide, preferably trimethoprim, and a nitroimidazole, preferably metronidazole; wherein when said composition C2 comprises or, alternatively, consists of an alpha blocker or alpha-lytic, said alpha blocker or alpha-lytic is selected from among doxazosin, terazosin, tamsulosin, silodosin; wherein when said composition C2 comprises or, alternatively, consists of a urinary antispasmodic, said urinary antispasmodic is oxybutynin; wherein when said composition C2 is a 5-alpha reductase inhibitor, said 5-alpha reductase inhibitor is selected from among finasteride and dutasteride; wherein when said composition C2 comprises or, alternatively, consists of an NSAID (Nonsteroidal anti-inflammatory drug), said NSAID is ibuprofen; wherein when said composition C2 comprises or, alternatively, consists of an antimycotic, said antimycotic is selected from among fluconazole, terconazole, clotrimazole, miconazole and butoconazole; wherein when said active ingredient C2 comprises or, alternatively, consists of a plant product (botanicals), said plant product is selected from among the root of nettle, ginkgo, Echinacea, goldenrod, strawberry tree, bearberry, highbush blueberry, rye and Serenoa repens.

14. A composition in a form for topical administration, comprising:
(i) a mixture M comprising or alternatively consisting of beclomethasone or a derivative thereof, wherein said derivative is beclomethasone prodrug, and, optionally,
(ii) at least one pharmaceutical grade additive and/or excipient.

15. The composition of claim 14, wherein the composition further comprises a glycosaminoglycan, collagen, an antibiotic, an alpha blocker/lytic, a urinary antispasmodic, a 5-alpha reductase inhibitor, an NSAID (Nonsteroidal anti-inflammatory drug), paracetamol, cortisone, an oestrogen, an antihistamine, an analgesic, an antimycotic, a plant product (botanicals) or a mixtures thereof.
